(19) United States
(12) Patent Application Publication
(10) Pub. No.: US 2025/0312504 A1
(43) Pub. Date: Oct. 9, 2025

(54) VOLATILE COMPOSITION DISPENSER FOR COMPOSITIONS COMPRISING A META-(C1-C4 ALKOXY) SALICYLALDEHYDE

(71) Applicant: The Procter & Gamble Company, Cincinnati, OH (US)

(72) Inventors: Bernardo M. AGUILERA-MERCADO, Kenwood, OH (US); Ryan Johnie SMITH, Cincinnati, OH (US); Ronald David TURNER, Walton, KY (US); Mu WANG, Cincinnati, OH (US); Philip John PORTER, Mason, OH (US)

(21) Appl. No.: 19/072,111

(22) Filed: Mar. 6, 2025

Related U.S. Application Data
(60) Provisional application No. 63/574,329, filed on Apr. 4, 2024.

(51) Int. Cl. A61L 9/12; A61L 101/36

(52) U.S. Cl. CPC A61L 9/125; A61L 2101/36; A61L 2202/14; A61L 2202/15

(57) ABSTRACT

A volatile composition dispenser includes a reservoir comprising a volatile composition; and an evaporative surface in fluid communication with the reservoir. The volatile composition comprises a first fragrance material that is a meta-(C1-C4 alkoxy)salicylaldehyde, and the first fragrance material is present in an amount of at least 0.0005%, by weight of the volatile composition. A process of treating an environment with a volatile composition dispenser includes providing a volatile composition dispenser and providing the meta-(C1-C4 alkoxy)salicylaldehyde to the environment. A weight loss of the volatile composition over less than 11 days ranges from 7% to 20%.

### TECHNICAL FIELD

The present disclosure relates to volatile composition dispensers including a volatile composition that includes a meta-(C1-C4 alkoxy)salicylaldehyde, such as ortho-ethylvanillin. The present disclosure further relates to processes of making and using such compositions.

### BACKGROUND

Consumer products, such as volatile composition dispensers, that have vanilla-scented fragrances are very popular. Such scents can be provided by compounds in the vanillin family, such as vanillin and ethylvanillin. Because ethylvanillin is perceived as having a greater intensity than vanillin, ethylvanillin may even be preferred to save raw material costs, carbon footprint and/or formulation space.

However, these materials can present challenges to the manufacturer and/or the consumer. For example, due to relatively high melting points, high boiling points, and/or low vapor pressures, the materials are associated with various processing, formulation, and/or performance issues, such as the tendency to crystallize at ambient temperatures. Furthermore, the materials may be characterized by relatively low solubility in certain commercially useful solvents such as isopropyl myristate (IPM), dipropylene glycol (DPG), dipropylene glycol methyl ether (DPM), tripropylene glycol methyl ether (TPM), making them inconvenient to process or formulate into products or intermediate compositions.

There is a need for improved vanilla-scented consumer products, compositions, and related processes that address these issues.

### SUMMARY

According to a first example of the present disclosure, a volatile composition dispenser includes: a reservoir comprising a volatile composition; and an evaporative surface in fluid communication with the reservoir, wherein: the volatile composition comprises a first fragrance material that is a meta-(C1-C4 alkoxy)salicylaldehyde, and the first fragrance material is present in an amount of at least 0.0005%, by weight of the volatile composition.

According to a second example of the present disclosure, a volatile composition dispenser includes a reservoir comprising a volatile composition; and an evaporative surface in fluid communication with the reservoir, wherein: the volatile composition comprises: a first fragrance material that is a meta-(C1-C4 alkoxy)salicylaldehyde; and a second fragrance material selected from the group consisting of vanillin, ethylvanillin, or a combination thereof, the first fragrance material is present in an amount of at least 0.0005%, by weight of the volatile composition, and the weight ratio of the first fragrance material to the second fragrance material is from 100:0 to 1:99.

According to a third example of the present disclosure, a process of treating an environment with a volatile composition dispenser includes providing a volatile composition dispenser including a reservoir comprising a volatile composition, and an evaporative surface in fluid communication with the reservoir. The volatile composition comprises a first fragrance material that is a meta-(C1-C4 alkoxy)salicylaldehyde; and the first fragrance material is present in an amount of at least 0.0005%, by weight of the volatile composition. The process further includes providing the meta-(C1-C4 alkoxy)salicylaldehyde to the environment, wherein a weight loss of the volatile composition over less than 11 days ranges from 7% to 20%.

### BRIEF DESCRIPTION OF THE DRAWINGS

The following detailed description of specific examples of the present disclosure can be best understood when read in conjunction with the following drawings, where like structure is indicated with like reference numerals and wherein:

FIG. 1 is a perspective view of a volatile composition dispenser, in accordance with examples of the present disclosure.

FIG. 2 is a profile view of a volatile composition dispenser, in accordance with examples of the present disclosure.

FIG. 2A is a cut-away view of a volatile composition dispenser, in accordance with examples of the present disclosure.

FIG. 3 is a graphical representation of weight loss over time, in accordance with examples of the present disclosure.

FIG. 4 is a graphical representation of weight loss over time, in accordance with examples of the present disclosure.

FIG. 5 is a graphical representation of weight loss over time, in accordance with examples of the present disclosure.

FIG. 6 is a collation of photographs of crystallization and lack of crystallization within volatile composition dispensers, in accordance with examples of the present disclosure.

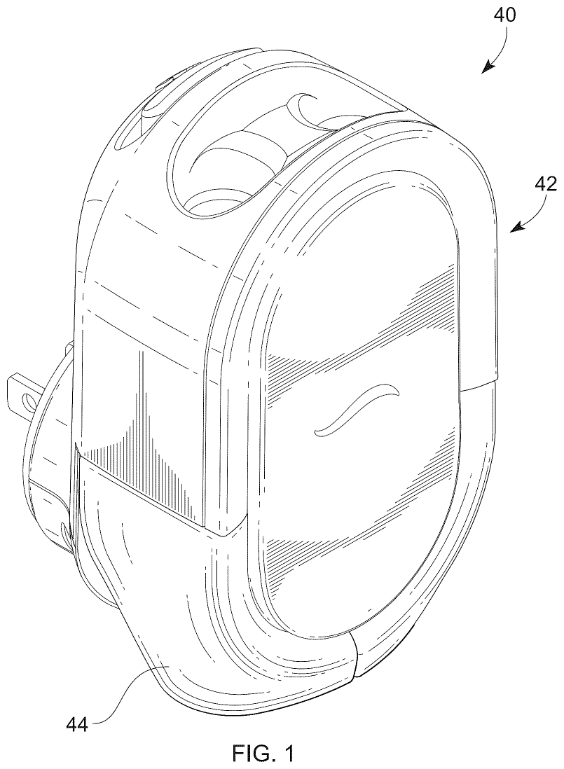

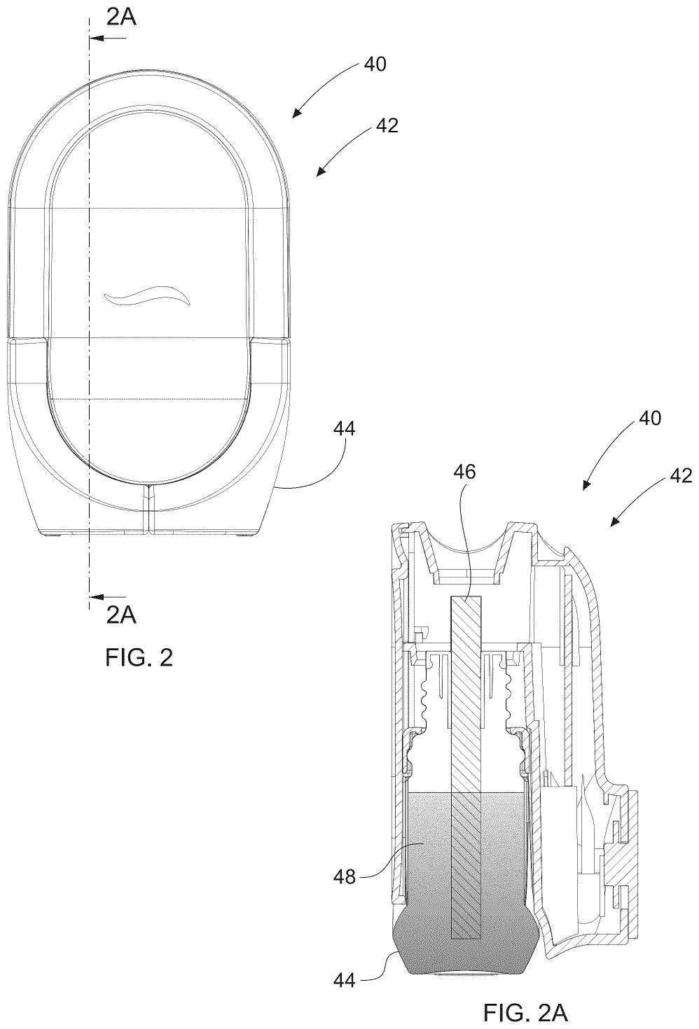

### DETAILED DESCRIPTION

The present disclosure relates to volatile composition dispensers including a volatile composition that includes meta-(C1-C4 alkoxy)salicylaldehyde. These fragrance materials have isomeric structures and scents that are similar to the more commonly used vanilla-scented materials (e.g., vanillin and/or ethylvanillin).

However, as described in more detail, the materials have certain advantageous characteristics that can make them more attractive to product manufacturers. For example, relatively lower melting temperatures can make them easier to pump through a pipe at lower temperatures, thereby resulting in lower energy costs to the manufacturer. Lower boiling points and/or higher vapor pressures enable them to volatilize more easily and be perceived by a consumer under normal usage conditions. Additionally, they are generally more soluble than their isomeric counterpart in common carriers or solvents, such as isopropyl myristate, dipropylene glycol methyl ether, and/or tripropylene glycol methyl ether, making them more convenient to formulate, particularly at relatively higher, scent character defining, levels.

The fragrance materials, compositions, consumer products, and related processes are discussed in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can include, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein, “consumer products” can include fine fragrance products or devices generally intended to be used or consumed in the form in which it is sold. Such products include but are not limited to products for and/or methods relating to treating fabrics, hard surfaces and any other surfaces in the area of fabric and home care, including: air care including air fresheners and scent delivery systems, fabric conditioning (including softening and/or freshening)

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all examples of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Volatile Composition Dispensers

It is contemplated that the volatile composition dispenser 40 may be configured for use in a variety of applications to deliver the volatile composition to the air and/or ultimately to a surface. The volatile composition dispenser 40 may be configured in various ways. For example, the volatile composition dispenser 40 may be configured as an electrical wall plug volatile composition dispenser 40 having a housing 42, a reservoir 44 containing a volatile composition 48, and a delivery engine that may be used to transport the volatile composition 48 to an evaporative surface 46.

The term “volatile compositions” as used herein, refers to a material that includes a vaporizable material. The term “volatile compositions,” thus includes (but is not limited to) compositions that are included entirely of a single volatile material. The terms “volatile materials,” “aroma,” “fragrance,” and “scents,” as used herein, include, but are not limited to pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile compositions including, but not limited to perfumes, aromatic materials, and scented materials, will often include one or more volatile materials (which may form a unique and/or discrete unit included of a collection of volatile materials). It should be understood that the term “volatile composition” refers to compositions that have at least one volatile component, and it is not necessary for all of the component materials of the volatile composition to be volatile. The volatile compositions described herein may, thus, also have non-volatile components. It should also be understood that when the volatile compositions are described herein as being “emitted,” this refers to the volatilization of the volatile components thereof and does not require that the non-volatile components thereof be emitted. The volatile compositions of interest herein can be in any suitable form including, but not limited to, solids, liquids, gels, encapsulates, and combinations thereof.

The reservoir 44 may include any suitable type of container and can be made of any suitable material. Suitable materials for construction of the reservoirs include, but are not limited to glass and plastic. The reservoir 44 may be constructed of any type of container that is suitable for holding volatile compositions. The reservoir 44 may be part of the housing 42, or may be a separate component that is removably joined to a portion of the volatile composition dispenser 40 such as the housing 42. It is also possible for a single reservoir 44 to hold more than one type of volatile material. Such a reservoir 44 could, for instance, have two or more compartments for volatile materials (not shown).

The delivery engine may include an evaporative surface 46. In such a configuration, the delivery engine may be placed next to one or more evaporative assistance elements, such as a heater or fan to volatilize the volatile composition 48 into the air. The evaporative assistance elements may surround, or at least partially surround, the evaporative surface 46.

In other examples, instead of evaporating the volatile composition 48 from an evaporative surface 46 of the delivery engine, the delivery engine may transport the volatile composition 48 to a separate evaporative surface (not shown). The evaporative surface may be configured as a porous or semi-porous substrate, a bowl or plate, including a plastic, glass, or metal bowl or plate, and combinations thereof.

The evaporative surface 46 may be configured in various ways. For example, the evaporative surface 46 may be in the form of a wick, membrane, gel, wax, porous or semi-porous substrate, including a felt pad. In a volatile composition dispenser 40 including more than one delivery engine associated with the same or different reservoirs (not shown), the delivery engines may be the same or may be different.

If the volatile composition dispenser utilizes a wick as the evaporative surface 46, the wick may be configured to have various different shapes and sizes. For example, the wick may have a cylindrical or an elongate cube shape. The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, at least 4 mm, at least 5 mm, at least 6 mm, or at least 7 mm. A wick may exhibit a density. The wick density may be in the range of about 0.100 grams/cm³ (“g/cc”) to about 1.0 g/cc.

A wick may include a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE or other polyolefins. The wick may be made from a plastic material such as polyethylene or a polyethylene blend.

meta-(C1-C4 alkoxy)salicylaldehyde

The compositions, products, and processes described herein include a meta-(C1-C4 alkoxy)salicylaldehyde compound. As described in more detail below, it is believed that such materials can provide a vanilla-like scent to compositions and products while also having certain advantageous characteristics that facilitate more convenient processing and/or improved performance.

As described herein, the meta-(C1-C4 alkoxy)salicylaldehyde compound is described as a (first) fragrance material. However, it is understood that, at acceptable levels and where appropriate, the compounds may also be used as flavoring materials.

The basic structure of a meta-(C1-C4 alkoxy)salicylaldehyde compound is provided below as Formula I:

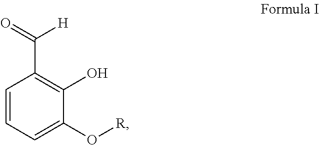

where R is a C1-C4 alkyl group (e.g., having from one to four carbon atoms). In other words, the —O—R group is the “C1-C4 alkoxy” group of the meta-(C1-C4 alkoxy)salicylaldehyde.

The C1-C4 alkoxy group includes from one to four carbon atoms. Due to structural similarities to vanillin and ethylvanillin, the alkoxy group is preferably a C1-C2 alkoxy group having from one to two carbon atoms (e.g., R is a C1-C2 alkyl group). More preferably, the group is a C2 alkoxy group that has two carbon atoms (e.g., R is a C2 alkyl group). It may also be preferred that the group is a C1 alkoxy group that has one carbon atom (e.g., R is a C1 alkyl group). It may also be preferred that the alkoxy group is a C2-C4 alkoxy group that has from two to four carbon atoms. The meta-(C1-C4 alkoxy)salicylaldehyde materials of the present disclosure may include compounds that have different alkoxy groups (e.g., mixtures of compounds having C1 alkoxy groups with other compounds having C2 alkoxy groups).

The C1-C4 alkyl group may be linear or branched, preferably the C1-C4 alkyl group is linear.

When the meta-(C1-C4 alkoxy)salicylaldehyde is a meta-(C1 alkoxy)salicylaldehyde, it is also known as ortho-vanillin. Ortho-vanillin has the following structure, shown as Formula II:

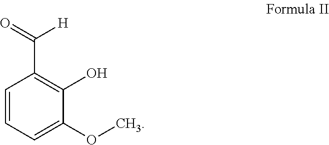 [SMILES: COc1cccc(C=O)c1O]

Other names for ortho-vanillin include: 2-hydroxy-3-methoxybenzaldehyde; 3-methoxysalicylaldehyde; or simply o-vanillin. Ortho-vanillin is identified by CAS No. 148-53-8. Ortho-vanillin, having the hydroxyl group at the ortho- position, is an isomer of vanillin, which has the hydroxyl group at the para- position.

When the meta-(C1-C4 alkoxy)salicylaldehyde is a meta-(C2 alkoxy)salicylaldehyde, it is also known as ortho-ethylvanillin. Ortho-ethylvanillin has the following structure, shown as Formula III:

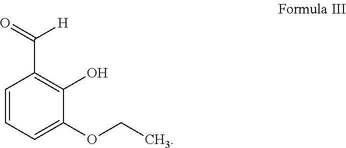 [SMILES: CCOc1cccc(C=O)c1O]

Other names for ortho-ethylvanillin include: novovanillin; 2-hydroxy-3-ethoxybenzaldehyde; 3-ethyoxysalicylaldehyde; or simply o-ethylvanillin. Ortho-ethylvanillin is identified by CAS No. 492-88-6. Ortho-ethylvanillin, having the hydroxyl group at the ortho-position, is an isomer of ethylvanillin, which has the hydroxyl group at the para- position.

As described further in the Examples section below, ortho-vanillin (i.e., meta-(C1 alkoxy)salicylaldehyde) and ortho-ethylvanillin (i.e., meta-(C2 alkoxy)salicylaldehyde) are characterized by certain characteristics, including melting point, boiling point, and LogP, that may make them desirable substitutes for, or co-ingredients with, vanillin and/or ethylvanillin.

### Volatile Compositions

The present disclosure relates to volatile compositions that include a meta-(C1-C4 alkoxy)salicylaldehyde. Typically, the volatile compositions will also include a treatment adjunct.

The volatile composition may include a first fragrance material that is a meta-(C1-C4 alkoxy)salicylaldehyde, where the first fragrance material is present in an amount of at least 0.0005%, by weight of the consumer product, and a treatment adjunct.

Advantageously, the properties of the meta-(C1-C4 alkoxy)salicylaldehydes described herein may allow them to be incorporated into consumer products at relatively higher levels than their isomeric counterparts. For example, because the materials of the present disclosure are relatively more soluble in common solvents or carriers (such as isopropyl myristate) than their isomeric counterparts, relatively greater amounts can be dissolved or otherwise formulated while maintaining adequate stability.

The first fragrance material may have a vapor pressure at 25° C. of greater than 8×10⁻⁴ Torr, more preferably greater than 2×10⁻³ Torr, and most preferably greater than 5×10⁻³ Torr. In examples, the first fragrance material may have a vapor pressure at 25° C. of from 8×10⁻⁴ Torr to 1×10⁻¹ Torr, from 2×10⁻³ Torr to 1×10⁻¹ Torr, from 5×10⁻³ Torr to 1×10⁻¹ Torr, from 8×10⁻³ Torr to 1×10⁻¹ Torr, from 1×10⁻² Torr to 1×10⁻¹ Torr, from 8×10⁻³ Torr to 5×10⁻² Torr, from 2×10⁻³ Torr to 5×10⁻² Torr, from 5×10⁻³ Torr to 5×10⁻² Torr, from 8×10⁻³ Torr to 5×10⁻² Torr, from 1×10⁻² Torr to 5×10⁻² Torr, from 8×10⁻⁴ Torr to 3×10⁻² Torr, from 2×10⁻³ Torr to 3×10⁻² Torr, from 5×10⁻³ Torr to 3×10⁻² Torr, from 8×10⁻³ Torr to 3×10⁻² Torr, from 1×10⁻² Torr to 3×10⁻² Torr, from 8×10⁻³ Torr to 2×10⁻² Torr, from 2×10⁻³ Torr to 2×10⁻² Torr, from 5×10⁻³ Torr to 2×10⁻² Torr, from 8×10⁻³ Torr to 2×10⁻² Torr, from 1×10⁻² Torr to 2×10⁻² Torr, or any values within the foregoing ranges or any ranges created thereby.

The first fragrance material may have a melting point of less than 80° C., more preferably less than 77° C., and most preferably less than 70° C. or any values within the foregoing ranges or any ranges created thereby. In examples, the first fragrance material may have a melting point of from 40° C. to 80° C., from 40° C. to 77° C., from 40° C. to 75° C., from 40° C. to 72° C., from 40° C. to 70° C., from 40° C. to 68° C., from 40° C. to 66° C., from 50° C. to 80° C., from 50° C. to 77° C., from 50° C. to 75° C., from 50° C. to 72° C., from 50° C. to 70° C., from 50° C. to 68° C., from 50° C. to 66° C., from 55° C. to 80° C., from 55° C. to 77° C., from 55° C. to 75° C., from 55° C. to 72° C., from 55° C. to 70° C., from 55° C. to 68° C., from 55° C. to 66° C., from 60° C. to 80° C., from 60° C. to 77° C., from 60° C. to 75° C., from 60° C. to 72° C., from 60° C. to 70° C., from 60° C. to 68° C., from 60° C. to 66° C., from 62° C. to 80° C., from 62° C. to 77° C., from 62° C. to 75° C., from 62° C. to 72° C., from 62° C. to 70° C., from 62° C. to 68° C., from 62° C. to 66° C., from 64° C. to 80° C., from 64° C. to 77° C., from 64° C. to 75° C., from 64° C. to 72° C., from 64° C. to 70° C., from 64° C. to 68° C., from 64° C. to 66° C., or any values within the foregoing ranges or any ranges created thereby.

The volatile composition may include the first fragrance material (e.g., the meta-(C1-C4 alkoxy)salicylaldehyde) in an amount of at least 0.0005%, preferably at least 0.001%, more preferably at least 0.01%, preferably in an amount of at least 0.1%, preferably at least 0.5%, more preferably at least 1%, by weight of the volatile composition. It is preferred to have a certain minimum amount of the first fragrance material present in order to deliver the desired olfactory performance.

The volatile composition may include the first fragrance material (e.g., the meta-(C1-C4 alkoxy)salicylaldehyde) in an amount of up to about 99%, preferably up to an amount of about 50%, preferably up to about 25%, more preferably up to about 10%, even more preferably up to about 5%, even more preferably up to about 1%, by weight of the volatile composition. It may be preferred to have a certain maximum amount or limit of the first fragrance material present in order to include other adjuncts or fragrance materials

The volatile composition may include the first fragrance material (e.g., the meta-(C1-C4 alkoxy)salicylaldehyde) in an amount of from about 0.01% to about 99%, preferably from about 0.1% to about 50%, more preferably from about 0.1% to about 30%, more preferably from about 0.5% to about 10%, more preferably from about 1% to about 5%, by weight of the volatile composition. The volatile composition may include the first fragrance material in an amount of from 0.0005% to 10%, from 0.0005% to 7%, from 0.0005% to 5%, from 0.0005% to 3%, from 0.0005% to 2%, from 0.0005% to 1%, from 0.001% to 10%, from 0.001% to 7%, from 0.001% to 5%, from 0.001% to 3%, from 0.001% to 2%, from 0.001% to 1%, from 0.01% to 10%, from 0.01% to 7%, from 0.01% to 5%, from 0.01% to 3%, from 0.01% to 2%, from 0.01% to 1%, from 0.1% to 10%, from 0.1% to 7%, from 0.1% to 5%, from 0.1% to 3%, from 0.1% to 2%, from 0.1% to 1%, from 0.5% to 10%, from 0.5% to 7%, from 0.5% to 5%, from 0.5% to 3%, from 0.5% to 2%, from 0.5% to 1%, from 1% to 10%, from 1% to 7%, from 1% to 5%, from 1% to 3%, from 1% to 2%, from 2% to 10%, from 2% to 7%, from 2% to 5%, from 2% to 3%, or any values within the foregoing ranges or any ranges created thereby.

The first fragrance materials (e.g., meta-(C1-C4 alkoxy)salicylaldehyde), particularly C1 and/or C2 alkoxy materials (e.g., ortho-vanillin and/or ortho-ethylvanillin), may be known to be in the presence of their isomeric counterparts (e.g., vanillin and/or ethylvanillin) when synthetically produced. However, the meta-(C1-C4 alkoxy)salicylaldehyde materials are typically treated as undesirable impurities that should be removed. In contrast, the present disclosure affirmatively seeks to formulate these materials into products and compositions due to their desirable characteristics. Thus, it may be desirable to formulate products and/or compositions that include certain weight ratios of the presently disclosed materials to their isomeric counterparts, including at weight ratios that are greater than 1:1.

For example, the volatile composition may include: a first fragrance material that is a meta-(C1-C4 alkoxy)salicylaldehyde; an optional second fragrance material selected from the group of vanillin, ethylvanillin, or a combination thereof; and a treatment adjunct; wherein the weight ratio of the first fragrance material to the second fragrance material, if present, is from 100:0 to 1:99.

The weight ratio of the first fragrance material to the second fragrance material (i.e., vanillin, ethylvanillin, or mixtures thereof) may be from 100:0 to 1:99, preferably from 100:0 to 25:75, preferably from 100:0 to 50:50, more preferably from 100:0 to 51:49, more preferably from 100:0 to 75:25, more preferably from 100:0 to 90:10, more preferably from 100:0 to 95:5, more preferably from 100:0 to 99:1.

It may be particularly preferred to control the weight ratio of the meta-(C1-C4 alkoxy)salicylaldehyde to its isomeric counterpart. For example, the weight ratio of ortho-vanillin to vanillin may be from 100:0 to 1:99, preferably from 100:0 to 25:75, preferably from 100:0 to 50:50, more preferably from 100:0 to 51:49, more preferably from 100:0 to 75:25, more preferably from 100:0 to 90:10, more preferably from 100:0 to 95:5, more preferably from 100:0 to 99:1. The weight ratio of ortho-ethylvanillin to ethylvanillin may be from 100:0 to 1:99, preferably from 100:0 to 25:75, preferably from 100:0 to 50:50, more preferably from 100:0 to 51:49, more preferably from 100:0 to 75:25, more preferably from 100:0 to 90:10, more preferably from 100:0 to 95:5, more preferably from 100:0 to 99:1.

It may be preferred to formulate only the presently disclosed materials and to leave out their isomeric counterparts. For example, the volatile composition may be substantially free of a second fragrance material selected from the group of vanillin, ethylvanillin, and a combination thereof. In particular, the volatile composition may include ortho-vanillin and be substantially free of vanillin. Additionally or alternatively, the volatile composition may include ortho-ethylvanillin and be substantially free of ethylvanillin.

As described above, the volatile composition includes a first fragrance material that is a meta-(C1-C4 alkoxy)salicylaldehyde. Preferably, the first fragrance material is a meta-(C1-C2 alkoxy)salicylaldehyde (i.e., ortho-vanillin, ortho-ethylvanillin, or a mixture thereof). More preferably, the first fragrance material is a meta-(C2 alkoxy)salicylaldehyde (i.e., ortho-ethylvanillin).

The alkoxy group in the first fragrance material may be a linear or branched alkoxy group, preferably a linear alkoxy group.

For convenient availability, it may be preferred that the first fragrance material is synthetically prepared. For environmental reasons, it may be preferred that the first fragrance material is naturally derived. When vanillin and/or ethylvanillin are present, it may be preferred for the first fragrance material to be naturally derived and present in a relatively large quantity. For example, the first fragrance material may be present in a weight ratio to a second fragrance, where the second fragrance material is selected from the group of vanillin, ethylvanillin, and a combination thereof, where the weight ratio is equal to or greater than 1:1.

The first fragrance material may be present as a free oil in the volatile composition, or it may be part of a perfume delivery system. For example, the meta-(C1-C4 alkoxy)salicylaldehyde may be encapsulated, or otherwise associated with (e.g., embedded in), a delivery particle. For example, the meta-(C1-C4 alkoxy)salicylaldehyde may be in the core of a core-shell delivery particle. The meta-(C1-C4 alkoxy)salicylaldehyde may be covalently or ionically bonded, preferably covalently bonded, to a carrier material, thereby forming a pro-fragrance material that releases the meta-(C1-C4 alkoxy)salicylaldehyde when the bond is broken, such as through exposure to water or light. Thus, the first fragrance material, e.g., the meta-(C1-C4 alkoxy)salicylaldehyde, may be part of a perfume delivery system, where the perfume delivery system is a delivery particle, a pro-fragrance material, or a combination thereof.

The fragrance or flavoring composition may include a second fragrance material that is different than the first fragrance material. The second fragrance material may be a perfume raw material. The term “perfume raw material” (or “PRM”) as used herein refers to compounds having a molecular weight of at least about 100 g/mol and which are useful in imparting an odor, fragrance, essence or scent, either alone or with other perfume raw materials. Typical PRMs include inter alia aromatic and/or aliphatic alcohols, ketones, aldehydes, esters, ethers, carboxylic acids, nitriles, nitrites and alkenes, such as terpene, and alkynes. A listing of common PRMs can be found in various reference sources, for example, “Perfume and Flavor Chemicals”, Vols. I and II; Steffen Arctander Allured Pub. Co. (1994) and “Perfumes: Art, Science and Technology”, Miller, P. M. and Lamparsky, D., Blackie Academic and Professional (1994).

The second fragrance material may be vanillin, ethylvanillin, or a mixture thereof. That being said, it may be particularly preferred to control the weight ratio of the meta-(C1-C4 alkoxy)salicylaldehyde to its isomeric counterpart. For example, the weight ratio of ortho-vanillin to vanillin may be from 100:0 to 1:99, preferably from 100:0 to 25:75, preferably from 100:0 to 50:50, more preferably from 100:0 to 51:49, more preferably from 100:0 to 75:25, more preferably from 100:0 to 90:10, more preferably from 100:0 to 95:5, more preferably from 100:0 to 99:1. The weight ratio of ortho-ethylvanillin to ethylvanillin may be from 100:0 to 1:99, preferably from 100:0 to 25:75, preferably from 100:0 to 50:50, more preferably from 100:0 to 51:49, more preferably from 100:0 to 75:25, more preferably from 100:0 to 90:10, more preferably from 100:0 to 95:5, more preferably from 100:0 to 99:1.

The second fragrance material may be selected from the group consisting of any fragrance material that is suitable for the intended product or end use. As non-limiting, illustrative examples, suitable second fragrance materials are provided below in Table A. The second fragrance material may be selected from the group provided in Table A, or combinations thereof.

TABLE A

| PRM | CAS |
|---|---|
| ALPHA DAMASCONE | 24720-09-0 |
| ALPHA PINENE | 80-56-8 |
| ALPHA-TERPINOLENE | 586-62-9 |
| AMBRONAT | 6790-58-5 |
| ANISIC ALDEHYDE | 123-11-5 |
| BENZYL ACETATE | 140-11-4 |
| BETA GAMMA HEXENOL | 928-96-1 |
| BETA PINENE | 127-91-3 |
| CARYOPHYLLENE EXTRA | 87-44-5 |
| CIS 3 HEXENYL ACETATE | 3681-71-8 |
| CITRONELLOL | 106-22-9 |
| COUMARIN | 91-64-5 |
| CYMAL | 103-95-7 |
| DECYL ALDEHYDE | 112-31-2 |
| DELTA DAMASCONE | 57378-68-4 |
| DIHYDRO MYRCENOL | 18479-58-8 |
| DIMETHYL OCTANOL | 106-21-8 |
| ETHYL MALTOL | 4940-11-8 |
| ETHYL-2-METHYL BUTYRATE | 7452-79-1 |
| GAMMA DECALACTONE | 706-14-9 |
| GAMMA TERPINENE COEUR | 99-85-4 |
| GERANIOL | 106-24-1 |
| GERANYL ACETATE | 105-87-3 |
| HABANOLIDE | 111879-80-2 |
| HELIOTROPIN | 120-57-0 |
| HEXYL ACETATE | 142-92-7 |
| HEXYL CINNAMIC ALDEHYDE | 101-86-0 |
| IONONE BETA | 14901-07-6 |
| IONONE GAMMA METHYL | 127-51-5 |
| ISO E SUPER OR WOOD | 68155-67-9 |
| LAEVO TRISANDOL | 28219-61-6 |
| LIGUSTRAL-1 | 68039-49-6 |
| LIMONENE | 5989-27-5 |
| LINALOOL | 78-70-6 |
| LINALYL ACETATE | 51685-40-6 |
| METHYL ANTHRANILATE | 134-20-3 |
| METHYL DIHYDRO JASMONATE | 24851-98-7 |
| METHYL IONONE ALPHA | 127-42-4 |
| METHYL PHENYL CARBINYL ACETATE | 93-92-5 |
| MYRCENE | 123-35-3 |
| N-BETA-METHYL IONONE ISOMER | 79-70-9 |
| NEROL | 106-25-2 |
| NERYL ACETATE | 141-12-8 |
| NONALACTONE | 63357-97-1 |
| OCIMEME | 3779-61-1 |
| PARA CYMENE | 99-87-6 |
| PHENYL ETHYL ALCOHOL | 60-12-8 |
| SABINENE | 3387-41-5 |
| UNDECALACTONE | 104-67-6 |
| VERDOX | 88-41-5 |

In order to provide a full and desirable olfactory experience, the volatile composition may include three or more fragrance materials. The volatile composition may further include a third fragrance material. The volatile composition may include from two to about fifty fragrance materials, preferably from two to about thirty fragrance materials. For simplicity and ease of manufacturing, it may be desirable to limit the number of components of the volatile composition. For example, the volatile composition may include from two to about twenty, or to about fifteen, or to about ten, fragrance materials.

As described above, the volatile composition may include a treatment adjunct. The treatment adjunct material may provide a benefit in the intended end-use of a composition, or it may be a processing and/or stability aid. The precise nature of the treatment adjunct(s), and levels of incorporation thereof, will depend on the physical form of the composition and the nature of the operation for which it is to be used. One of ordinary skill will be able to select appropriate materials and levels for a given product or application.

Suitable treatment adjuncts may include surfactants, conditioning actives, deposition aids, rheology modifiers or structurants, bleach systems, stabilizers, builders, chelating agents, dye transfer inhibiting agents, dispersants, enzymes, enzyme stabilizers, catalytic metal complexes, polymeric dispersing agents, clay and soil removal/anti-redeposition agents, brighteners, suds suppressors, silicones, hueing agents, aesthetic dyes, neat perfume, additional perfume delivery systems, structure elasticizing agents, carriers, hydrotropes, processing aids, anti-agglomeration agents, coatings, formaldehyde scavengers, pigments, or mixtures thereof.

The treatment adjunct may include a carrier. Such carriers may be hydrophilic (e.g., water) or hydrophobic (e.g., an oil) in nature. The presently described meta-(C1-C4 alkoxy)salicylaldehyde are relatively hydrophobic in nature, and may be conveniently coupled with a hydrophobic carrier. Particularly useful carriers in volatile compositions include, but are not limited to, isopropyl myristate (IPM), dipropylene glycol (DPG), dipropylene glycol methyl ether (DPM), tripropylene glycol methyl ether (TPM), triethyl citrate (TEC), dimethyl adipate, or mixtures thereof. Thus, the volatile composition may include a carrier, where the carrier is preferably IPM, DPM, or mixtures thereof. Preferably, the carrier includes isopropyl myristate.

Isopropyl myristate, as well as other hydrophobic carriers, may be particularly useful when the meta-(C1-C4 alkoxy)salicylaldehyde is encapsulated in certain core-shell delivery particles, such as those having acrylate-based or chitosan-based shells. The cores of the delivery particles may include both the isopropyl myristate and the meta-(C1-C4 alkoxy)salicylaldehyde, preferably C1-C2 alkoxy, more preferably C2 alkoxy.

### Additional Fragrance Materials:

The fragrance compositions disclosed herein may also include additional fragrance materials. Some non-limiting examples of such additional fragrance materials are listed in Table B below. At least one of the additional fragrance materials listed in Table B may be utilized alone in a volatile composition, or utilized in a volatile composition in combination with any other fragrance materials disclosed herein.

TABLE B

| IUPAC Name | CAS Number |
|---|---|
| 1-[1-(2-ethylbutyl)cyclohexyl]propan-1-one | |
| 1-(3,5-dimethyl-1-adamantyl)ethanone | 40430-57-7 |
| 1-(1-adamantyl)butan-1-one | 24556-00-1 |
| 1-(1-adamantyl)ethanone | 1660-04-4 |
| 1-(1-adamantyl)heptan-1-one | 32665-99-9 |
| 1-(1-adamantyl)hexan-1-one | 32665-97-7 |
| 1-(1-adamantyl)propan-1-one | 1660-05-5 |
| 1-but-2-enyl-4-methylbenzene | 21003-53-2 |
| 1-(lambda1-methoxy)naphthalene | 2216-69-5 |
| 1-pentoxy-4-propoxybenzene | 2650262-16-9 |
| 3-[(2,5-diethylfuran-3-yl)disulfanyl]-2,5-diethylfuran | 2650948-13-1 |
| 3-[(2,5-dimethylfuran-3-yl)disulfany1]-2,5-dimethylfuran | 28588-73-0 |
| 2-ethyl-3-[(2-ethyl-5-methylfuran-3-yl)disulfanyl]-5- | |
| methylfuran | |
| 2-methyl-3-[(2-methylfuran-3-yl)disulfanyl]furan | 28588-75-2 |
| 1,4-dipentoxybenzene | 32527-64-3 |
| 1,4-diethynylbenzene | 30700-96-0, 26713-43-9, |
| | 935-14-8 |
| 1-ethyl-4-prop-2-enylbenzene | 18640-62-5 |
| 1-methyl-4-prop-2-enylbenzene | 3333-13-9 |
| 1-butoxy-4-pentoxybenzene | 2650948-13-1 |
| 1-ethoxy-4-pentoxybenzene | 2225737-76-6 |
| 1-ethyny1-4-methylbenzene | 766-97-2, 34807-69-7 |
| 1-methoxyanthracene | 61128-86-7 |
| 1-methoxynaphthalene | 2216-69-5 |
| 2-butylcyclohexan-1-one | 1126-18-7 |
| 2-ethylcyclohexan-1-one | 4423-94-3 |
| 2-methylbenzo[g][1,3]benzoxazole | 20686-65-1 |
| 2-nonylcyclohexan-1-one | 16556-72-2 |
| 2-pentylcyclohexan-1-one | 280575-02-2, 32362-97-3 |
| 2-propylcyclohexan-1-one | 64870-42-4, 94-65-5 |
| 3-(p-tolyl)prop-1-en-1-one | |
| 3,5,5-trimethylcyclohex-2-en-1-ol | 114818-71-2, 470-99-5 |
| 3,5-diethylbenzaldehyde | 81698-95-5 |
| 3-butyl-5,5-dimethylcyclohex-2-en-1-ol | 133969-86-5 |
| 3-(disulfanyl)-2,5-dimethylfuran | |
| 3-ethoxy-3-oxo-2-phenylpropanoic acid | 17097-90-4 |
| 3-ethyl-5,5-dimethylcyclohex-2-en-1-ol | 31952-19-9 |
| 3-ethyl-5-methylbenzaldehyde | 3132-93-2 |
| 3-ethylbenzaldehyde | 34246-54-3 |
| 5,5-dimethyl-3-(2-methylpropyl)cyclohex-2-en-1-ol | 1478030-64-6 |
| 3-propan-2-ylbenzaldehyde | 34246-57-6 |
| 4-(3-oxopropyl)benzaldehyde | 90392-95-3 |
| 4-but-3-enylbenzaldehyde | 137658-83-4 |
| [4-(cyclopropanecarbonyl)phenyl] formate | |
| 4-heptoxybenzaldehyde | 27893-41-0 |
| 4-pentoxyphenol | 18979-53-8 |
| 4-butylbenzaldehyde | 1200-14-2 |
| 4-carboxy-2-methoxycarbonylphenolate | |
| 4-hydroxy-3-methoxycarbonylbenzoic acid | 41684-11-1 |
| 4-(2-methylpropyl)benzaldehyde | 40150-98-9 |
| 4-propylbenzaldehyde | 28785-06-0 |
| 5,5-dimethyl-3-propylcyclohex-2-en-1-ol | 118465-28-4 |
| 5-ethylbenzene-1,3-dicarbaldehyde | 118792-76-0 |
| 5-methoxynaphthalen-2-ol | 150712-57-5 |
| 9-methoxyanthracene | 2395-96-2 |
| prop-2-enylbenzene | 128961-80-8, 57807-91-7, |
| | 25988-53-8, 300-57-2 |
| butyl 2-oxocyclohexane-1-carboxylate | 64486-45-9 |
| butyl 2-oxocyclopentane-1-carboxylate | 6627-69-6 |
| cyclopropyl-(4-ethoxyphenyl)methanone | 75343-44-1 |
| cyclopropyl-(4-propan-2-yloxyphenyl)methanone | 1152833-63-0 |
| cyclopropyl-(4-methoxyphenyl)methanone | 7152-03-6 |
| cyclopropyl-(4-propoxyphenyl)methanone | 1226182-72-4 |
| decyl 2-oxocyclohexane-1-carboxylate | |
| diethyl 2-(4-ethylphenyl)propanedioate | 29148-28-5 |
| diethyl 2-(4-methylphenyl)propanedioate | 29148-27-4 |
| diethyl 2-phenylpropanedioate | 83-13-6 |
| dimethyl 4-hydroxybenzene-1,3-dicarboxylate | 5985-24-0 |
| ethyl 2-oxocyclohexane-1-carboxylate | 1655-07-8, 4840-68-0 |
| ethyl 2-oxocyclopentane-1-carboxylate | 611-10-9 |
| ethyl 3,4-dimethoxybenzoate | 3943-77-9 |
| ethynylbenzene | 536-74-3, 120851-94-7, |
| | 25038-69-1 |
| hexyl 2-oxocyclohexane-1-carboxylate | 1631736-32-7 |
| hexyl 2-oxocyclopentane-1-carboxylate | 72845-82-0 |
| methyl 2,5-dioxocyclopentane-1-carboxylate | 2825007-84-7 |
| methyl 2-hydroxy-5-(hydroxymethyl)benzoate | 183430-63-9 |
| methyl 2-hydroxy-5-(methoxymethyl)benzoate | 861315-31-3 |
| methyl 2-oxocyclopentane-1-carboxylate | 10472-24-9 |
| methyl 3,4-dimethoxybenzoate | 2150-38-1, 3943-74-6 |
| methyl 4-hydroxy-3-methoxybenzoate | 3943-74-6 |
| methyl 5-(hydroxymethyl)pyrazine-2-carboxylate | 1262803-64-4 |
| methyl 5-(methoxymethyl)pyrazine-2-carboxylate | 1388149-35-6 |
| methyl 5-formylpyrazine-2-carboxylate | 710322-57-9 |
| methyl 5-methylpyrazine-2-carboxylate | 41110-33-2 |
| methyl 2-oxocyclopentane-1-carboxylate | 10472-24-9 |
| methyl pyrazine-2-carboxylate | 6164-79-0 |
| propyl 2-oxocyclohexane-1-carboxylate | 29537-13-1 |
| tert-butyl 4-(2-aminoethyl)-4-methylpiperidine-1-carboxylate | 946517-83-5 |
| tert-butyl 4,4-diethylpiperidine-1-carboxylate | 1498576-45-6 |
| tert-butyl 4,4-dimethylpiperidine-1-carboxylate | 1252572-05-6 |
| tert-butyl 4-ethyl-4-methylpiperidine-1-carboxylate | 1495020-67-1 |
| tert-butyl 6-azaspiro[2.5]octane-6-carboxylate | 955028-67-8 |
| ethenyl 3,4-dimethoxybenzoate | 1644148-22-0 |

### Process of Treatment

The present disclosure also relates to a process of treating an environment with a volatile composition dispenser having a volatile composition including a meta-(C1-C4 alkoxy)salicylaldehyde, preferably a meta-(C1-C2 alkoxy)salicylaldehyde, more preferably a meta-(C2 alkoxy)salicylaldehyde, as described in more detail above.

The process of treatment includes the step of providing the meta-(C1-C4 alkoxy)salicylaldehyde to an environment. Suitable environments may include interior spaces such as a room in a residential dwelling, a commercial property, or an industrial setting.

The process may include the step of providing the meta-(C1-C4 alkoxy)salicylaldehyde to an environment, preferably an interior environment, as part of a consumer product according to the present disclosure. The step of providing the meta-(C1-C4 alkoxy)salicylaldehyde may occur via a spraying process or through diffusion.

The process of treating an environment a volatile composition dispenser having a volatile composition as previously described may result in a volatile composition weight loss of from 7% to 20%, 7% to 15%, 7% to 13%, 10% to 20%, 10% to 15%, 10% to 13%, 11% to 13%, or approximately 12% in less than 11 days, less than 10 days, less than 9 days, less than 8 days, less than 7 days, or about 6 days.

### Use

The present disclosure also relates to the use of a meta-(C1-C4 alkoxy)salicylaldehyde, preferably a meta-(C1-C2 alkoxy)salicylaldehyde, more preferably a meta-(C2 alkoxy)salicylaldehyde, to provide a vanilla-type fragrance to a consumer product, a surface, or an environment. Such materials, products, surfaces, and environments are discussed in more detail above.

### Combinations

A. A volatile composition dispenser comprising:
a reservoir comprising a volatile composition; and
an evaporative surface in fluid communication with the reservoir, wherein:
   the volatile composition comprises a first fragrance material that is a meta-(C1-C4 alkoxy)salicylaldehyde, and
   the first fragrance material is present in an amount of at least 0.0005%, by weight of the volatile composition.
      B. The volatile composition dispenser as disclosed in A, wherein the volatile composition further comprises a treatment adjunct.
      C. The volatile composition dispenser as disclosed in B, wherein the treatment adjunct comprises a carrier comprising isopropyl myristate (IPM), dipropylene glycol (DPG), dipropylene glycol methyl ether (DPM), tripropylene glycol methyl ether (TPM), triethyl citrate (TEC), dimethyl adipate, or combinations thereof.
      D. The volatile composition dispenser as disclosed in any one of A-C, wherein the first fragrance material is a meta-(C1-C2 alkoxy)salicylaldehyde.
      E. The volatile composition dispenser as disclosed in any one of A-D, wherein the first fragrance material is a meta-(C2 alkoxy)salicylaldehyde.
      F. The volatile composition dispenser as disclosed in any one of A-E, wherein the alkoxy group in the first fragrance material is a linear alkoxy group.
      G. The volatile composition dispenser as disclosed in any one of A-F, wherein the first fragrance material is present in the volatile composition in an amount of at least 0.001%, by weight of the volatile composition.
      H. The volatile composition dispenser as disclosed in any one of A-G, wherein the first fragrance material is synthetically prepared.
      I. A volatile composition dispenser comprising:
a reservoir comprising a volatile composition; and
an evaporative surface in fluid communication with the reservoir, wherein:
the volatile composition comprises:
   a first fragrance material that is a meta-(C1-C4 alkoxy)salicylaldehyde; and
   a second fragrance material selected from the group consisting of vanillin, ethylvanillin, or a combination thereof,
   the first fragrance material is present in an amount of at least 0.0005%, by weight of the volatile composition, and
   the weight ratio of the first fragrance material to the second fragrance material is from 100:0 to 1:99.
      J. The volatile composition dispenser as disclosed in I, wherein the weight ratio of the first fragrance material to the second fragrance material, is from 100:0 to 25:75.
      K. The volatile composition dispenser as disclosed in I or J, wherein the volatile composition further comprises a treatment adjunct.
      L. The volatile composition dispenser as disclosed in K, wherein the treatment adjunct comprises a carrier comprising isopropyl myristate (IPM), dipropylene glycol (DPG), dipropylene glycol methyl ether (DPM), tripropylene glycol methyl ether (TPM), triethyl citrate (TEC), dimethyl adipate, or combinations thereof.
      M. The volatile composition dispenser as disclosed in any one of I-L, wherein the first fragrance material is a meta-(C1-C2 alkoxy)salicylaldehyde.
      N. The volatile composition dispenser as disclosed in any one of I-M, wherein the first fragrance material is a meta-(C2 alkoxy)salicylaldehyde.
      O. The volatile composition dispenser as disclosed in any one of I-N, wherein the alkoxy group in the first fragrance material is a linear alkoxy group.
      P. The volatile composition dispenser as disclosed in any one of I-O, wherein the first fragrance material is present in the volatile composition in an amount of at least 0.001%, by weight of the volatile composition.
      Q. The volatile composition dispenser as disclosed in any one of I-P, wherein the first fragrance material is synthetically prepared.
      R. A process of treating an environment with a volatile composition dispenser comprising:
providing a volatile composition dispenser comprising:
   a reservoir comprising a volatile composition, and
   an evaporative surface in fluid communication with the reservoir, wherein:
   the volatile composition comprises a first fragrance material that is a meta-(C1-C4 alkoxy)salicylaldehyde; and
   the first fragrance material is present in an amount of at least 0.0005%, by weight of the volatile composition, and
providing the meta-(C1-C4 alkoxy)salicylaldehyde to the environment, wherein a weight loss of the volatile composition over less than 11 days ranges from 7% to 20%.
   S. The process as disclosed in R, wherein:
the weight loss of the volatile composition over less than 9 days ranges from 7% to 15%; and
no crystallization occurs within the volatile composition dispenser.
   T. The process as disclosed in R or S, wherein:
the first fragrance material is a meta-(C2 alkoxy)salicylaldehyde; and
the first fragrance material is present in the volatile composition in an amount of at least 0.001%, by weight of the volatile composition.

### Test Methods

It is understood that the test methods disclosed in the Test Methods section of the present application should be used to determine the respective values of the parameters of Applicant's claimed subject matter as claimed and described herein.

### Solubility Method

Solutions of vanillin (121-33-5), ethylvanillin (121-32-4), and o-ethylvanillin (492-88-6) are prepared at 8.00E⁻² g/L in IPM. Each of these is used to make five additional sequential serial two-fold dilutions with IPM giving a set of six solutions for each solute. The absorbance values for solutions of vanillin and ethyl vanillin (at 300 nm), and of o-ethylvanillin (at 343 nm) are used to create standard calibration curves for each molecule (linear regression correlation coefficients R²≥0.998).

Mixtures of each compound at 1, 5, 10, 15, 20, 25, and 30 weight percent in IPM are prepared by adding the solid compound to IPM, sonicating at 50° C. for 1 hour and allowing to cool to room temperature for 3 hours. Some of these solutions are saturated, i.e. are comprised of both dissolved and undissolved compound. Saturated solutions of each compound are then filtered to remove undissolved compound and each filtrate was diluted 1:10,000 in IPM. The absorbance values of the filtrates of vanillin and of ethyl vanillin (at 300 nm), and of o-ethylvanillin (at 343 nm) are used to calculate the concentration based on the calibration curves, and the maximum concentration values are considered to be the solubility limits for vanillin, ethyl vanillin, and o-ethylvanillin in IPM.

### Headspace Method

Perfume headspace data is collected for various materials according to the method provided below.

The partition coefficients are calculated by determining the amount of PRM in the headspace above a solution with a known concentration using a GC/FID-MS (Agilent 7890 and 5977A MSD) with a thermal desorption unit (Gerstel MPS2) and Dynamic Headspace Sampler (Gerstel DHS). This technique allows for automated headspace collections using the MultiPurposeSampler (MPS). The PRMs of interest are prepared at a known concentration in isopropyl myristate (IPM) or dipropylene glycol methyl ether (DPM), and 1 mL is pipetted in a 20 mL headspace vial.

DHS Conditions: The sample is tempered in the unit at 30° C. A helium stream at 10 mls/min purges 20 mL of headspace containing the analytes above the sample onto the sorbent tube (TDU) containing 25 mg of TenaxTA 35/60 (Restek cat #25701).

TDU conditions: The sorbent tube is desorbed with a program starting at 30° C. solvent vent for 2 min then ramped at 720° C./min to 270° C. and held for 8 min. The analytes are refocused on a glass bead liner at −60° C. then desorbed at a rate of 12° C./s to 270° C. onto the GC column.

GC/MS conditions: Agilent 122-5532UI column of 30 m×250 m×0.25 m and a flow rate of 1.5 mL/min is utilized. A temperature program of 50° C. to 280° C. with a 2 min hold and a ramp rate of 12° C./min is suitable. Scan or SIM mode MSD parameters with a solvent delay for the DPM are applied to achieve appropriate detection.

Analytical standards can be obtained from Sigma Aldrich, St. Louis, MO and BioSynth, UK. A stock solution is prepared at approximately 0.5 g/L. The stock solution is then diluted to 4 to 5 concentrations ranging from approximately 0.05 g/L to 0.00005 g/L. These standard solutions are then analyzed by GC/MS. This analysis is performed by using the same TDU desorption and GC/MS conditions described above with a luL liquid injection onto the TDU tube instead of the DHS collection. The amount injected on column and the area responses of the target m/z are utilized to create a calibration plot with a linear fit.

Quantification of the headspace is performed by the PRM target m/z area response of the perfume component and the linear equation of the calibration plot.

### Examples

The examples provided below are intended to be illustrative in nature and are not intended to be limiting.

### Example 1. Comparison of Materials and Characteristics/Physical Properties

The following tables compare the structures and various characteristics of materials according to the present disclosure with their isomeric counterparts. Specifically, Table 1 compares comparative material vanillin to ortho-vanillin (aka, meta-(C1 alkoxy)salicylaldehyde). Table 2 compares comparative material ethylvanillin to ortho-ethylvanillin (aka, meta-(C2 alkoxy)salicylaldehyde).

TABLE 1

| | | ortho-Vanillin |
|---|---|---|
| | Vanillin | (aka, meta-(C1 |
| | (comp.) | alkoxy)salicylaldehyde) |
| Structure | 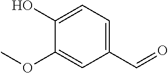 | 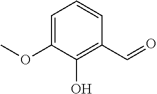 |
| CAS No. | 121-33-5 | 148-53-8 |
| Melting Point | 80-81° C. | 44.5° C. |
| Boiling Point | 284-285° C. | 265.5° C. |
| ClogP | 1.208 ± 0.272 | 1.600 ± 0.279 |
| vapor pressure* | 1.94 × 10⁻³ | 5.56 × 10⁻³ |
| (25° C., Torr) | | |

TABLE 2

| | | ortho-Ethylvanillin |
|---|---|---|
| | Ethylvanillin | (aka, meta-(C2 |
| | (comp.) | alkoxy)salicylaldehyde) |
| Structure | 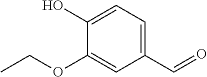 | 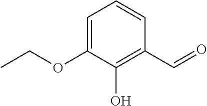 |
| CAS No. | 121-32-4 | 492-88-6 |
| Melting Point | 77.5° C. | 64-65° C. |
| Boiling Point | 285° C. | 263-264° C. |
| ClogP | 1.718 ± 0.272 | 2.109 ± 0.279 |
| Solubility (g/mL) in | 0.038 | 0.073 |
| IPM | | |
| vapor pressure* | 8.84 × 10⁻⁴ | 1.20 × 10⁻² |
| (25° C., Torr) | | |

*Vapor Pressure @ 25° C. is a theoretical value predicted by a Physicochemical Properties model simulator from ACD/Labs.

As shown in Tables 1 and 2, compared to its isomer (vanillin and ethylvanillin, respectively, having the hydroxyl in the para- position), the ortho- version of each material is characterized by a lower melting point, which is associated with a decreased tendency to crystallize and which indicates that lower amounts of energy are required to melt and flow the material through a pipe.

Furthermore, the ortho- version of each material is characterized by a lower boiling point, which tends to be associated with a higher vapor pressure. Such materials are likely to volatilize more easily and may therefore be perceived at lower levels in a consumer product. This in turn allows them to be formulated at lower levels, thereby saving formulation space.

Additionally, the ortho- version of each material is characterized by a higher ClogP than its para- counterpart, indicating that the ortho- materials are relatively more hydrophobic and/or may partition into hydrophobic solvents (such as IPM) more efficiently. With regard to ethylvanillin vs. ortho-ethylvanillin, this is shown by the differences in solubility in IPM.

### Example 2. Headspace Data

Perfume headspace data is collected for various materials according to the Headspace Method provided above in the Test Methods section. The Air/Solvent partition coefficients are determined and indexed accordingly (e.g., where the partition coefficient for ethylvanillin is set at 1.00).

Results for vanillin, ethylvanillin, and ortho-ethylvanillin in isopropyl myristate (PM) and dipropylene glycol methyl ether (DPM) are provided below in Table 3 and 4, respectively.

TABLE 3

| | | | | Air/IPM | Ethylvanillin |
|---|---|---|---|---|---|
| | | Concentration | Head- | partition coef | Indexed Air/IPM |
| | CAS | in IPM | space | (ng/mL in air per | partition |
| Sample | No. | (ng/mL) | (ng/mL)* | ng/mL in IPM)* | coefficient* |
| Vanillin | 121-33-5 | 2.07E+06 | 0.0563 | 2.73E−08 | 1.40 |
| (comp.) | | | | | |
| Ethylvanillin | 121-32-4 | 2.06E+06 | 0.0402 | 1.95E−08 | 1.00 |
| (comp.) | | | | | |
| ortho- | 492-88-6 | 2.10E+06 | 0.2521 | 1.21E−07 | 6.17 |
| Ethylvanillin | | | | | |

*average of two replicates.

TABLE 4

| | | | | Air/DPM | Ethylvanillin |
|---|---|---|---|---|---|
| | | Concentration | Head- | partition coef | Indexed Air/DPM |
| | CAS | in DPM* | space | (ng/mL in air per | partition |
| Sample | No. | (ng/mL) | (ng/mL)* | ng/mL in DPM)* | coefficient* |
| Vanillin | 121-33-5 | 1.1E+07 | 0.1288 | 1.21E−08 | 4.88 |
| (comp.) | | | | | |
| Ethylvanillin | 121-32-4 | 1.1E+07 | 0.0262 | 2.45E−09 | 1.00 |
| (comp.) | | | | | |
| ortho- | 492-88-6 | 1.1E+07 | 2.4678 | 2.31E−07 | 93.7 |
| Ethylvanillin | | | | | |

*average of three replicates.

Overall, ortho-ethylvanillin goes into the headspace about 6.2× more than its structural isomer ethylvanillin out of IPM, a common solvent used in perfume delivery particles, and about 94× more than ethylvanillin out of DPM. The increase in volatility of ortho-ethylvanillin compared to ethylvanillin is advantageous because a higher fraction of ortho-ethylvanillin will be found in the air than the consumer product. Therefore, ortho-ethylvanillin will be more likely to be perceived at lower levels in consumer products or at greater intensity at equal levels in consumer products.

### Example 3

Ethylvanillin (EV), vanillin (V), and o-ethylvanillin (OEV) were each dissolved in dipropylene glycol methyl ether (DPM) (34590-94-8) at 2, 5 and 10 wt. % in an ultrasonic bath at 50° C. Each solution (24 mL) was added to a P&G electronic air freshener device and plugged in to a standard 120v outlet. The devices were inspected and weighed daily for 4 weeks. The mass of each device was no longer recorded after the device stopped losing weight.

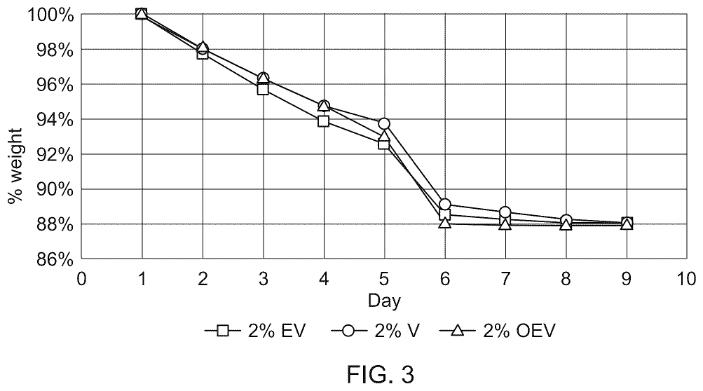

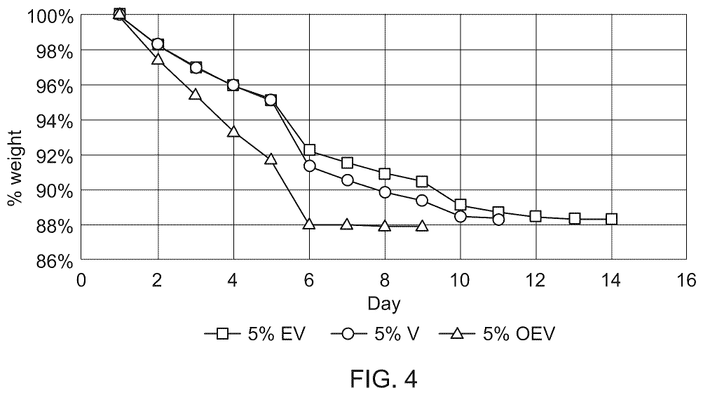

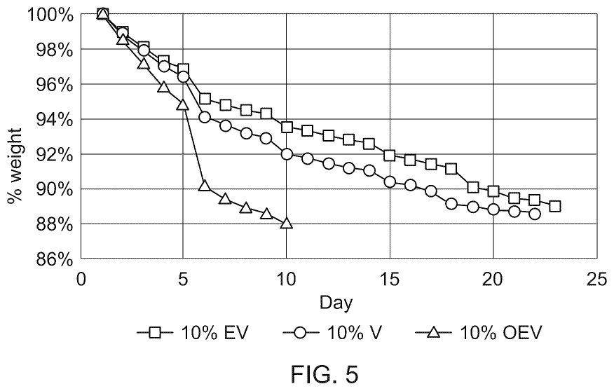

FIGS. 3-5 show the % weight loss of each solution in the P&G electronic air freshener device. FIG. 3 shows the % weight loss over time of the 2% solutions of EV, V, and OEV. FIG. 4 shows the % weight loss over time of the 5% solutions of EV, V, and OEV. FIG. 5 shows the % weight loss over time of the 10% solutions of EV, V, and OEV.

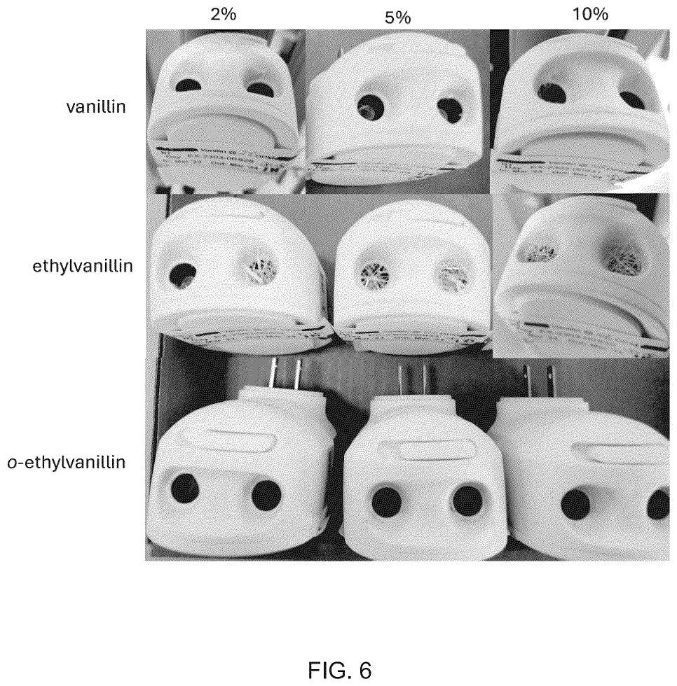

Referring to FIG. 6, crystals were seen to form in the EV and V air freshener device over time. However, no crystals formed in the air freshener devices with OEV solutions, as shown in FIG. 6. Referring to FIGS. 3-5, the OEV solutions exhibit a steeper weight loss rate than the EV and V solutions. This is most noticeable in the 5% and 10% solutions. This increased rate of weight loss as compared to the EV and V solutions is believed to be due to the lack of crystallization in the OEV air freshener devices.

It is contemplated that the OEV solution does not form crystals due to its chemical structure (in which the hydroxyl group is adjacently located between the carbonyl and the ethoxy groups) hindering the intramolecular hydrogen bond interactions. This results in a lower propensity of OEV molecules to closely pack together when compared to V and EV. This reduction in intermolecular forces of OEV when compared to V and EV is also manifested as OEV having a lesser melting point and greater vapor pressure than V or EV. A lesser melting point typically implies lower crystallinity. Compared to V and EV, OEV has a relatively lesser melting point (as shown in Tables 1 & 2 above (V=80-81° C., EV=77.5° C., and OEV=64-65° C.)) and therefore lead to less crystal formation. In addition, the vapor pressure of OEV is relatively greater than the vapor pressure of EV and V. Vapor pressure is a physical property that describes a material's thermodynamic tendency to evaporate. A relatively greater vapor pressure indicates an increased propensity to evaporate. Compared to V and EV, OEV has a relatively greater vapor pressure (as shown in Tables 1 & 2 above (V=1.94×10⁻³ Torr, EV=8.84×10⁻⁴ Torr, and OEV=1.20×10⁻² Torr)) and therefore may evaporate faster from a volatile fragrance composition thereby resulting in a more rapid weight loss of the volatile composition. It is contemplated that the relatively faster evaporation of OEV (as compared to V and EV) may result in a greater fraction of OEV reaching a consumer's nose and imparting a more enjoyable fragrance experience as compared to if the volatile composition included V or EV.

Once V, EV, or OEV have evaporated from the liquid fragrance composition in the air freshener device there are two possible outcomes: 1) the molecules can condense on the device (crystallize) or 2) the molecules can fully escape the device and remain in the gaseous phase. Compared to OEV, a greater fraction of V and EV molecules condense on the device resulting in undesired crystal formation due to EV and V having a relatively lower vapor pressure, relatively greater melting point, and/or having greater intermolecular forces (a greater number of hydrogen bonds) than OEV. Compared to EV and V, a lesser fraction of OEV molecules condense on the device and no crystals form due to OEV having a relatively greater vapor pressure, having relatively lesser melting point, and/or having lesser intermolecular forces (hydrogen bonds) as compared to EV and V.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to anything disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A volatile composition dispenser comprising:
a reservoir comprising a volatile composition; and
an evaporative surface in fluid communication with the reservoir,
wherein:
the volatile composition comprises a first fragrance material that is a meta-(C1-C4 alkoxy)salicylaldehyde, and
the first fragrance material is present in an amount of at least 0.0005%, by weight of the volatile composition.

2. The volatile composition dispenser of claim 1, wherein the volatile composition further comprises a treatment adjunct.

3. The volatile composition dispenser of claim 2, wherein the treatment adjunct comprises a carrier comprising isopropyl myristate (IPM), dipropylene glycol (DPG), dipropylene glycol methyl ether (DPM), tripropylene glycol methyl ether (TPM), triethyl citrate (TEC), dimethyl adipate, or combinations thereof.

4. The volatile composition dispenser of claim 1, wherein the first fragrance material is a meta-(C1-C2 alkoxy)salicylaldehyde.

5. The volatile composition dispenser of claim 1, wherein the first fragrance material is a meta-(C2 alkoxy)salicylaldehyde.

6. The volatile composition dispenser of claim 1, wherein the alkoxy group in the first fragrance material is a linear alkoxy group.

7. The volatile composition dispenser of claim 1, wherein the first fragrance material is present in the volatile composition in an amount of at least 0.001%, by weight of the volatile composition.

8. The volatile composition dispenser of claim 1, wherein the first fragrance material is synthetically prepared.

9. A volatile composition dispenser comprising:
a reservoir comprising a volatile composition; and
an evaporative surface in fluid communication with the reservoir, wherein:
the volatile composition comprises:
a first fragrance material that is a meta-(C1-C4 alkoxy)salicylaldehyde; and
a second fragrance material selected from the group consisting of vanillin, ethylvanillin, or a combination thereof,
the first fragrance material is present in an amount of at least 0.0005%, by weight of the volatile composition, and
a weight ratio of the first fragrance material to the second fragrance material is from 100:0 to 1:99.

10. The volatile composition dispenser of claim 9, wherein the weight ratio of the first fragrance material to the second fragrance material is from 100:0 to 25:75.

11. The volatile composition dispenser of claim 9, wherein the volatile composition further comprises a treatment adjunct.

12. The volatile composition dispenser of claim 11, wherein the treatment adjunct comprises a carrier comprising isopropyl myristate (IPM), dipropylene glycol (DPG), dipropylene glycol methyl ether (DPM), tripropylene glycol methyl ether (TPM), triethyl citrate (TEC), dimethyl adipate, or combinations thereof.

13. The volatile composition dispenser of claim 9, wherein the first fragrance material is a meta-(C1-C2 alkoxy)salicylaldehyde.

14. The volatile composition dispenser of claim 9, wherein the first fragrance material is a meta-(C2 alkoxy)salicylaldehyde.

15. The volatile composition dispenser of claim 9, wherein the alkoxy group in the first fragrance material is a linear alkoxy group.

16. The volatile composition dispenser of claim 9, wherein the first fragrance material is present in the volatile composition in an amount of at least 0.001%, by weight of the volatile composition.

17. The volatile composition dispenser of claim 9, wherein the first fragrance material is synthetically prepared.

18. A process of treating an environment with a volatile composition dispenser comprising:
providing a volatile composition dispenser comprising:
a reservoir comprising a volatile composition, and
an evaporative surface in fluid communication with the reservoir, wherein:
the volatile composition comprises a first fragrance material that is a meta-(C1-C4 alkoxy)salicylaldehyde; and
the first fragrance material is present in an amount of at least 0.0005%, by weight of the volatile composition, and
providing the meta-(C1-C4 alkoxy)salicylaldehyde to the environment, wherein a weight loss of the volatile composition over less than 11 days ranges from 7% to 20%.

19. The process of claim 18, wherein:
the weight loss of the volatile composition over less than 9 days ranges from 7% to 15%; and
no crystallization occurs within the volatile composition dispenser.

20. The process of claim 18, wherein:
the first fragrance material is a meta-(C2 alkoxy)salicylaldehyde; and
the first fragrance material is present in the volatile composition in an amount of at least 0.001%, by weight of the volatile composition.
